(19) United States
(12) Patent Application Publication
(10) Pub. No.: US 2014/0227725 A1
(43) Pub. Date: Aug. 14, 2014

(54) COMPETITIVE S100A9 IMMUNOASSAYS

(71) Applicant: Magne Fagerhol, Oslo (NO)

(72) Inventor: Magne Fagerhol, Oslo (NO)

(73) Assignee: CALPRO AS, Lysaker (NO)

(21) Appl. No.: 14/127,492

(22) PCT Filed: Jun. 21, 2012
(86) PCT No.: PCT/EP2012/061972

§ 371 Date: Apr. 23, 2014

(30) Foreign Application Priority Data
Jun. 21, 2011 (NO) 20110895

(51) Int. Cl. G01N 33/68; G01N 33/574

(52) U.S. Cl. CPC G01N 33/6893; G01N 33/6896; G01N 33/57488
USPC 435/7.93; 436/501; 435/287.2; 422/401

(57) ABSTRACT

The present invention concerns a method for determining the concentration of calprotectin in a sample. A lateral flow test kit and test element using said method are also provided.

### FIELD OF INVENTION

The present invention concerns a competitive Enzyme-Linked Immunosorbent Assay (ELISA) and competitive lateral flow rapid tests (LFT) for detection of the proteins S100A9 and calprotectin.

### BACKGROUND OF INVENTION

Calprotectin belongs to the S100 family of proteins. The name derives from the fact that they are resistant to precipitation by ammonium sulphate so that they are soluble even in 100 percent saturated (thus 100S) solution. It is believed that they have evolved by a large number point mutation, but many amino acid sequence homologies remain. For this reason, some antibodies can bind to epitopes that are common for many or at least several S100 proteins. A common feature of these proteins is that they can bind calcium and zinc and thereby become resistant to enzymatic degradation; this is especially true for calprotectin. In the presence of calcium calprotectin will form dimers, while S100A12 (A12) will form oligomers, mostly dimers, tetramers and hexamers. Calprotectin is a heterotrimer consisting of two subunits called S100A9 (A9) and one called S100A8 (A8). Each of these subunits can bind two calcium molecules, i.e. a total of six per calprotectin molecule.

The subunits and their genes were fully sequenced in the late 1980's and thus are well known in the art. (Odink et al., “Two calcium-binding proteins in infiltrate macrophages of rheumatoid arthritis”, Nature, 1987 Nov. 5-11; 330 (6143): 80-2 Lagasse et al., “cloning and expression of two human genes encoding calcium-binding proteins that are regulated during myeloid differentiation,” Mol. Cell. Biol. 1988 June; 8(6): 2402-10, Andersson et al., “The leucocyte L1 Protein: identity with the cystic fibrosis antigen and the calcium-binding MRP-8 and MRP-14 macrophage components,” Scand. J. Immunol., August; 28(2): 241-5 (1988). Also the crystalline structures of the subunits have been determined, (Itou et al., “The crystal structure of human MRP14 (S100A9), a Ca(2+)-dependent regulator protein in inflammatory process.” J. Mol. Biol. 2002 Feb. 15; 316(2):265-76, Itou et al., “Expression, purification, crystallization and preliminary X-ray diffraction analysis of human calcium-binding protein MRP14 (S100A9)”. Acta Crystallogr. D Biol. Crystallogr. 2001 August; 57(pt 8): 1174-6, Moncrief et al., “Evolution of EF-hand calcium-modulated protein. I. Relationship based on amino acid sequence,” J. Mol. Evol., June; 30(6): 522-62 (1990), Raftery et al., “Overexpression, oxidative refolding, and zinc binding of recombinant forms of the murine S100 protein MRP14 (S100A9),” Protein Expr. Purif. 1999 March; 15(2): 228-35, Raftery et al., “Isolation of the murine S100 protein MRP14 (14 kDa migration-inhibitory-factor-related protein) from activated spleen cells: characterization of post-translation modifications and zinc binding,” Biochem. J., May 15; 316 (Pt1): 285-93 (1996), Loomans et al., “Histidine-based zinc-binding sequence and the antimicrobial activity of calprotectin,” J. Infect. Dis. March; 177(3): 812-4 (1998), Rety et al., “Structural basis of the Ca(2+)-dependent association between S100C(S100A11) and its targets, the N-terminal part of annexin I,” Structure Fold. Des. 2000 Feb. 15; 8(2): 175-84.

Both calprotectin and S100A12 are abundant in neutrophil granulocytes and monocytes and are released from these cells during inflammation, cell damage or cell death. They are therefore found in increased concentration in blood, other body fluids, secretions and excretions during inflammation for which they may be useful markers.

Calprotectin may be used as a marker for a number of diseases wherein excessive levels of calprotectin activity characterise the diseases. Such diseases include, but are not limited to, inflammatory bowel disease, rheumatoid arthritis, cystic fibrosis, inflammatory dermatosis, liver diseases, neurodegenerative diseases, Alzheimer's disease, dementia, multiple sclerosis and cancers.

An important prerequisite for quantitative immunoassays is that the analyte calibrators should have the same molecular configuration as that in the test sample. Calprotectin, a major protein in the cytosol of neutrophil granulocyte is a heterotrimer of one S100A8 and two S100A9 subunits. The structure of this protein in leukocyte extracts has been determined by electrophoresis, isoelectric focussing, ion exchange chromatography, gel filtration, circular dichroism, equilibrium dialysis and analytical ultracentrifugation, (Fagerhol & al., 1980, Dale et al., 1983; Naess-Andresen & a., 1995; Berntzen & Fagerhol, 1990; Berntzen & al., 1988; and reviewed in the book chapter by Fagerhol & al., 1990. In the presence of calcium in relatively low concentrations, for instance 2 mM, a dimeric form consisting of two trimers, is generated. By immunization of animals, for instance rabbits, antibodies against several epitopes of S100A8 and S100A9 will be generated. Even if the test antibodies react only with one epitope on each of the two subunits, the signal in immunoassays, for instance ELISA, will be stronger (theoretically doubled) if calprotectin is present as a double heterotrimer compared to the single heterotrimer in the absence of calcium. When an assay for calprotectin in stool samples was developed (Røseth & al., 1992), it was found that using a simple extraction buffer, for instance phosphate buffered saline, pH 7.4, only about 15 percent of the total calprotectin was found in the extract; the sample had to be re-extracted several times to obtain a close to 100% yield. When extracts were run on gel filtration (gel permeation chromatography), about 90% of the anti-calprotectin reactivity was found in the 100 to 1000 kDa region suggesting that in stool extracts, calprotectin is present mostly in high molecular size complexes. By use of an improved stool extraction buffer (Ton & al., Improved Assay for Fecal Calprotectin, Clin. Chem. Acta 292: 41-54 (2000)), the yield of calprotectin increased significantly to 41-70 100%, with a mean of about 78%. By running an extract prepared by that method on a gel filtration column using a buffer with calcium, calprotectin reactivity was found mostly in the 60 to 80 kDa fractions corresponding to the size of a double heterotrimer, see FIG. 1. In samples with lower yields, reactivity was also found in much higher molecular size fraction. Clearly, there are big differences between the native calprotectin purified from leukocytes and that in stool extracts and even between extracts from different individuals. Another important difference has been found, namely that calprotectin in stool samples does not react with a specific monoclonal antibody against the S100A8 subunit, in contrast to strong reactions against native as well as recombinant calprotectin. This should theoretically give falsely low values when stool extracts are run in immunoassays, for instance ELISA, using antibodies reacting both with S100A8 and S100A9 and native calprotectin in the calibrators. This has been supported by extensive experiments where antibodies, both monoclonal antibodies and polyclonal antibodies, have been tried. An additional source of error in estimates is that some epitopes on calprotectin may be altered or hidden in the large complexes referred to above.

Another source of error in measuring analytes such as calprotectin in immunoassays such as sandwich immunoassays, is the high dose hook effect. The high dose hook effect refers to measured levels of antigen displaying a significantly lower absorbance than the actual level present in a sample. This appears when a “single step” ELISA assay is used; i.e. the standards/samples are incubated together with the enzyme conjugated antibody. At a very high concentration of sample antigen most antigen binding sites on the conjugate will be occupied thereby preventing the formation of the “sandwich” that is subject to detection in the immunoassay. The “sandwich” consists of antigen bound to two antibody molecules; typically, one of these antibody molecules is unlabeled and bound to a solid support, while the other antibody molecule is labelled with a detectable label. The antigen-saturated detection antibodies in solution will be washed off giving a falsely low signal. A “hook” is observed by the standard curve dropping off at antigen concentrations above a certain level, for instance 10 000 ng/ml, when data is plotted as a signal versus antigen concentration. The possible existence of a high dose hook effect is very significant with respect to the use of calprotectin concentrations for the diagnosis or monitoring of inflammatory bowel disease, as, in the event a calprotectin concentration in a stool sample is high enough to trigger the high dose hook effect, it will be incorrectly measured as a low concentration of calprotectin. The risk of a hook effect in single step ELISA or rapid test for calprotectin is higher than for many other markers because levels in stools may be as high as 90 000 μg/L which is nearly 2000 times the upper normal limit. Errors caused by a hook effect will cause a failure to properly diagnose inflammatory bowel disease. The clinical consequences of this error are significant, because such patients would then be assumed not to have inflammatory bowel disease, but rather a less serious condition such as irritable bowel syndrome. They would then be given incorrect treatment and their inflammatory bowel disease would go untreated leading to complications that even may require surgery.

Calprotectin is recognized as the most practical and specific biomarker for the diagnosis, detection, or monitoring of inflammatory bowel disease. Although a number of assays for calprotectin are known, there is a necessity for an improved assay for calprotectin that is specific for calprotectin and has a wide dynamic range, such that it is not susceptible to interferences that can occur. Additionally, there is a necessity for an improved assay for calprotectin that is highly specific for calprotectin and can distinguish calprotectin from other closely related S100 proteins. Moreover, there is a necessity for an improved calprotectin assay that can give results much quicker than those presently available on the market, and can avoid a high dose hook effect.

### BRIEF DESCRIPTION OF THE DRAWINGS

FIG. 1 shows the presence of calprotectin reactivity in serum and a stool extract run on a column for gel permeation chromatography (GPC) capable of separating proteins according to their molecular size. In serum, calprotectin eluted corresponding to 60 to 80 kDa, i.e. close to that of albumin. However, some calprotectin molecules elute at about 100 kDa. In other stool extracts calprotectin eluted in high molecular size fractions corresponding to 100 to 1000 kDa or larger indicating significant heterogeneity in calprotectin in stools.

FIG. 2 shows a standard curve obtained when using a single specific S100A9 monoclonal antibody for coating of wells and a polyclonal antibody enzyme conjugate.

FIG. 3 shows the correlation between estimates of stool extracts using recombinant calprotectin or S100A9 as standards.

FIG. 4a shows a lateral flow test set-up. The set-up consists of a nitrocellulose strip (1), attached to a rigid, inert plastic membrane (2). A solution with of S100A9 is applied as a line across the strip in position (3). A line of donkey IgG is applied in position (4). When the test is performed the first end of the strip is put in vertical position into a microwell containing a mixture of sample and colloid gold labelled antibodies against S100A9 for the testline (3) and labelled antibodies against donkey IgG for the control line (4). After a few minutes the sample and antibodies has diffused upwards into the strip, binding of labelled antibodies will generate coloured line at position (3) for calprotectin/S100A9 and at position (4) for the control. (6) is a pad of absorbent material, e.g. water absorbent paper.

FIG. 4b shows an alternative lateral flow test set-up wherein an additional “sample pad” (5) containing dried labelled antibodies is attached to the first end of the strip, onto which samples can be applied while keeping the strip horizontally.

FIG. 5 shows the test and control lines on strips added S100A9 in different amounts together with labelled anti-S100A9 and labelled anti-donkey IgG. The staining intensity of the test line decreases with increasing concentration of S100A9 while the colour of the control line increases with increasing concentration of S100A9.

FIG. 6 shows a comparison of faecal calprotectin values obtained with the competitive S100A9 methods according to the invention and the original ELISA.

FIG. 7 shows a competitive lateral flow test for calprotectin.

FIG. 8 is a standard curve for competitive calprotectin lateral flow rapid test.

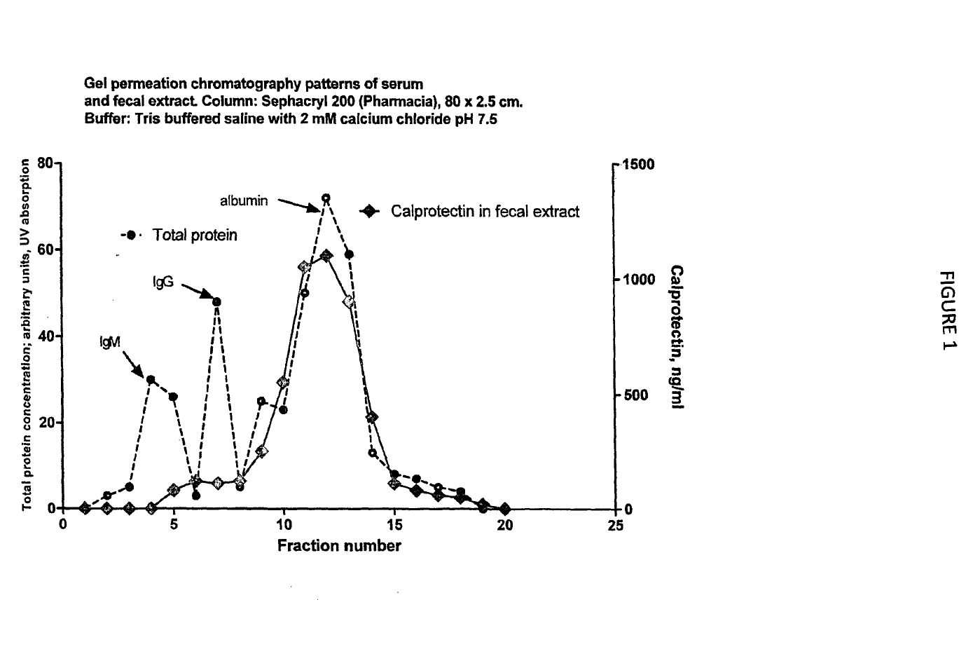

### SUMMARY OF THE INVENTION

This invention describes competitive immunoassay for detection and quantification of the leukocyte derived protein calprotectin and its subunit S100A9 in human or animal samples. The methods have wide assay ranges, give results within ten to 40 minutes, avoid hook effects and require a single monoclonal antibody. The latter has been carefully selected to react with an antigenic epitope present on calprotectin in stool extracts.

### DETAILED DESCRIPTION OF THE INVENTION

Surprisingly it has been found that some of the problems mentioned above might be solved by using antibodies against S100A9 only. An even more strict S100A9/anti-S100A9 methodology might be based upon a competitive immunoassay, for instance ELISA, where S100A9, rather than antibodies are used for capture, for instance for coating of microwells.

Satisfactory such a competitive assay gave a nice standard curve, values in stool extracts comparable to those obtained when using the original calprotectin ELISA (Røseth & al., 1992) and eliminated the problem of hook effect (antigen excess) typical of other “single step or incubation” methods. Furthermore, the system based on the ideas mentioned above has other advantages also: the assay takes only about 30 minutes compared to about three hours for standard ELISAs; the consumption of reagents is low; recombinant S100A9 can be used for capture (for instance coating of wells); recombinant calprotectin or S100A9, both in rich supply, can be used as calibrators, and selected monoclonal antibodies are also available in rich supply. The monoclonal antibodies must be selected by tests showing non-reactivity against S100A8 and S100A12, but good reactivity against a panel of stool extracts from a large number of IBD patients. Antibodies reacting with normal calprotectin and S100A9 but not with stool calprotectin molecules lacking some S100A9 epitopes in stool extracts must be avoided. An additional advantage of the system is that delayed addition of samples/calibrators to some wells will have less effect on the values compared to commonly used sandwich ELISAs. In these, wells are coated with antibodies and calibrators/samples are incubated first followed by washing and a second incubation with an enzyme labelled antibody, but it will take several minutes to add samples to all 96 wells in plate, so that those added late will have a shorter incubation period and therefore give a falsely low results.

Unexpectedly, it has been found that an improved immunoassay for calprotectin that is effective in determining the concentration of calprotectin in stool samples, such as would be used in clinical practice, and that is not subject to the high dose hook effect that can affect sandwich immunoassays, employs the principle of a competitive immunoassay using immobilized S100A9 and labelled anti-S100A9 antibody.

In such a competitive immunoassay, the S100A9 subunit of calprotectin in the sample competes with the immobilized S100A9 in the wells for binding of a limited amount of the labelled anti-S 100A9 antibody added. As only labelled anti-S 100A9 antibody bound to the immobilized S100A9 is eventually detected in such a competitive immunoassay, the higher the concentration of calprotectin in the sample, the less labelled antibody is bound to the solid support. A standard curve then can be constructed using appropriate calibrators; the standard curve will reflect the properties of a competitive immunoassay in which a lower signal detected indicates a higher concentration of analyte such as S100A9

The competitive immunoassay for calprotectin uses immobilized S100A9 on a solid support. Although the S100A9 can be isolated and purified from calprotectin in leukocyte extracts, this is technically demanding and requires non-standard purification methods (Berntzen H B & Fagerhol M K, L1, a major granulocyte protein; isolation of high quantities of its subunits, Scand J Clin Lab Invest 1990; 50(7):769-74), the use of recombinant S100A9 as described below has many advantages. Furthermore as an alternative, peptides with an amino acid sequence identical to that of the antigenic epitope on S100A9 can be synthesized and used for immobilization on the solid support. By creation of many different monoclonal antibodies, a monoclonal antibody can be chosen that will react with human as well as animal S100A9 since there are many inter species amino acid sequence homologies.

In the competitive immunoassay for calprotectin, the antibody that specifically binds the S100A9 can be a polyclonal antibody or a monoclonal antibody. However, as described below, monoclonal antibodies are preferred because they are much more uniform and available in large amounts allowing better standardization of commercial kits. It is preferred to use a monoclonal antibody that specifically binds the S100A9, i.e. without cross-reaction with related proteins like S100A8 or S100A12, and in particular binds to an epitope present on calprotectin in stool samples.

According to the present invention, various formats can be used in the performance of the assay. In one format, a platform similar to that typically employed for ELISA assays is used, designated herein as a “solid phase platform,” with the S100A9 being bound to the solid support and then sample and labelled antibody added together to the solid support after a washing step. The immobilized S100A9 and any free S100A9 in the sample are then allowed to react and compete for the limited quantity of labelled antibody added. After any S100A9 in the sample has been allowed to react and compete for binding of the labelled anti-S100A9 antibody with the immobilized S100A9 bound to the solid support, a washing step is then performed to remove unbound antibody. Labelled antibody bound to the solid support is then detected. Alternatives for this assay format are described below.

In another format, a lateral flow or flow through platform can be used as is generally known in the art; the lateral flow alternative platform is designated herein as a “lateral flow test (LFT).”, and the flow through is designated FTT. In the lateral flow platform format, immobilized S100A9 is bound at a defined detection zone in one portion of a test strip that is permeable to sample and to mobile labelled antibody. The sample and the mobile labelled antibody are then applied to the test strip and allowed to migrate through the test strip so that any S100A9 present in calprotectin in the sample competes for the binding to the anti-S100A9 antibody immobilized at the detection zone of the test strip. Various alternatives for applying the sample and the mobile labelled antibody are known in the art. The quantity of labelled antibody bound to the detection zone is then determined to provide an indication of the quantity of calprotectin in the sample. In the FTT alternative S100A9 is attached and immobilized on a permeable membrane, for instance nitrocellulose, with pores of a size large enough to allow reagents, including antibodies, to flow through during the procedure. A mixture of labelled anti-S100A9 and sample is applied on the membrane, and S100A9 or calprotectin in the sample or standard and the immobilized S100A9 will compete for binding to the labelled antibody. By choosing as suitable amount of such antibody, the amount that binds to the membrane will be inversely proportional to the amount of S100A9 or calprotectin in the sample. The label on the antibody can be colloid gold particles, colloid silver particles, stained particles of any suitable material for instance, but not limited to, latex, magnetic particles or particles that can be stimulated, for instance by exposure to UV light, to emit light that can be recorder by a suitable instrument. In the event that gold, silver or stained particles are used, the colour intensity of the detection zone can be recorder by a spectrophotometer. A standard curve for colour intensities given when calibrator with different S100A9 concentrations are tested can be generated and fed into a computer so that concentrations of A100A9 or calprotectin in samples can be estimated based on the staining intensity of the detection zone compared with those of the calibrators by aid of a suitable computer and computer program.

Although purified S100A9 from neutrophil granulocytes can be used as the immobilized S100A9, it is generally preferred to use recombinant S100A9. A recombinant S100A9 can be produced by methods well known in the art; typically a DNA sequence corresponding to the gene for S100A9 can by synthesized and cloned into cells, typically a microbe, which can be grown and produce S100A9. Both the amino acid sequence and DNA sequence of S100A9 is as mentioned before well known to the skilled in the art and freely available from Gene banks on the Internet.

Such prepared and purified recombinant S100A9 may be used in the preparation of monoclonal and polyclonal antibodies by standard techniques well known in the art.

In one aspect of the invention a monoclonal antibody to recombinant S100A9 is provided by use of methods generally known in the art.

Although isolated and purified S100A9 polypeptide can be used as antigen, it is generally preferred to use recombinant S100A9 polypeptide. A suitable recombinant S100A9 protein has the sequence

(SEQ ID NO: 1)
MTCKMSQLERNIETIINTFHQYSVKLGHPDTLNQGEFKELVRKDLQNFLK
KENKNEKVIEHIMEDLDTNADKQLSFEEFIMLMARLTWASHEKMHEGDEG
PGHHHKPGLGEGTP.

The genomic DNA sequence encoding S100A9 is

(SEQ ID NO: 2)
aaacactctgtgtggctecteggctttgacagagtgcaagacgatgactt
gcaaaatgtcgcagctggaacgcaacatagagaccatcatcaacaccttc
caccaatactctgtgaagctggggcacccagacaccctgaaccaggggga
attcaaagagctggtgcgaaaagatctgcaaaattttctcaagaaggaga
ataagaatgaaaaggtcatagaacacatcatggaggacctggacacaaat
gcagacaagcagctgagettcgaggagttcatcatgctgatggcgaggct
aacctgggcctcccacgagaagatgcacgagggtgacgagggccctggcc
accaccataagccaggcctcggggagggcaccccctaagaccacagtggc
caagatcacagtggccacggccacggccacagtcatggtggccacggcca
cagccactaatcaggaggccaggccaccctgcctctacccaaccagggcc
ccggggcctgttatgtcaaactgtettggctgtggggctaggggctgggg
ccaaataaagtctcttcctccaagtcaaaaaaaaaa.

The sequence of SEQ ID NO: 2 is derived from mRNA and therefore excludes introns possibly present in the genomic sequence. A DNA sequence encoding A9 that is suitable for expression in Escherichia coli is GGTACCATATGACCTGCAAAATGAGCCAGCTGGAACGTAACATTGAAACC ATCATCAACACCTTTCATCAGTATAGCGTGAAACTGGGCCATCCGGATAC CCTGAACCAGGGCGAATTTATGATCGAACACATCATGGAAGATCTGGATA CCAACGCGGATAAACAGCTGTCTTTCGAAGAAGAACTGGTGCGTAAAGAT CTGCAGAACTTCCTGAAAAAAGAAAACAAAAACGAAAAAGAATTTATTAT GCTGATGGCGCGTCTGACCTGGGCGAGCCATGAAAAAATGCATGAAGGCG ATGAAGGCCCGGGTCATCATCATAAACCGGGCCTGGGCGAAGGCACCCCG TGATAACTCG (SEQ ID NO: 3); this sequence is optimized for expression in E. coli.

When selecting a sequence for the recombinant preparation of or synthesis of peptide(s) the peptide must react with a monoclonal anti-S100A9. Said monoclonal antibody must also react with calprotectin/calprotectin complexes in samples, e.g. faecal extracts.

Another aspect the present invention provides a method for determining the concentration of calprotectin in a sample comprising the following steps:

i) coating a purified S100A9 onto a solid phase,
ii) reacting the S100A9 coated on the solid phase in step i) with a) a sample containing calprotectin and b) a labelled anti-S100A9 antibody,
iii) washing of the solid phase after the reaction to remove non-bound calprotectin, free labelled anti-S100A9 and calprotectin-labelled anti S100A9 antibody complexes.
iv) determining the quantity of labelled anti-S100A9 antibody bound to the solid phase to determine the concentration of calprotectin in the sample;
wherein the quantity of S100A9 coated onto the solid phase and the quantity of labelled anti-S 100A9 antibody are chosen such that the S100A9 coated onto the solid phase and any calprotectin in the sample compete for the labelled anti-S100A9 antibody such that the quantity of labelled anti-S100A9 antibody bound to the solid support is related inversely to the concentration of calprotectin in the sample.

The label of the labelled anti-S100A9 antibody can be any conventional label as known in the art. When the assay is performed using the solid phase platform as described above, in general, an enzyme label is used. When an enzyme label is used, the step of determining the quantity of labelled anti-S 100A9 antibody bound to the solid phase comprises the step of incubating the solid phase with a substrate for the enzyme of the enzyme-labelled anti-S100 antibody.

A number of suitable enzymes are known in the art for use in these assays. These enzymes include, but are not limited to, alkaline phosphatase, horseradish peroxidase, glucose 6-phosphate dehydrogenase, and β-galactosidase. Other enzyme labels are also known in the art. Such additional enzymes include, but are not necessarily limited to, acetate kinase, β-lactamase, glucose oxidase, firefly luciferase, laccase, Renilla luciferase, and xanthine oxidase. Enzyme-labelled antibodies can be prepared by methods known in the art such as covalent coupling procedures.

In many cases, the enzyme in the enzyme-labelled antibody produces a product that is detected and/or quantified photometrically, such as by spectroscopy. However, in some alternatives, the enzyme produces a product that is monitored and/or quantified by other means, such as detection and/or quantification of fluorescence, bioluminescence, or chemiluminescence.

Typically, the step of coating the solid support, for instance the interior of microwells used for ELISA, with S100A9 dissolved in Tris-buffered saline with from about 0.2 mM to 2 mM calcium present, such as calcium chloride. Preferably, the calcium concentration is from about 0.5 mM to about 1.5 mM, more preferred, the calcium concentration is about 1 mM. Typically, the S100A9 is incubated with the solid support at a concentration of from 0.4 μg/mL to about 2 μg/mL, preferably a concentration of from 0.6 μg/mL to about 2 μg/mL, more preferred a concentration of about 0.8 μg/mL.

In an embodiment of the invention the solid phase in step i) is coated with calprotectin, and particularly the solid phase is coated with a recombinant calprotectin.

Preferably, for the incubation of the solid support with the S100A9 to bind the S100A9 to the solid support, the solid support is covered with vapor tight adhesive plastic and stored at a temperature from about 0° C. to about 10° C., preferably about 5° C., for an incubation period of from about 6 hours to several weeks. Preferably, the incubation period is about 18 hours.

In one embodiment of the invention, subsequent to the step of binding the S100A9 polypeptide to the solid support, the solid support is washed with Tris-buffered saline with from about 0.2 mM to 2 mM calcium present, such as calcium chloride. Preferably, the calcium concentration is from about 0.5 mM to about 1.5 mM, more preferred about 1 mM.

In this embodiment, subsequent to the washing step, a conditioning step is typically performed on the solid support. In the conditioning step, the solid support is incubated with a solution of sucrose and bovine serum albumin containing a phosphate buffer, referred to herein as “SBP buffer.” Typically, the concentration of sucrose in the SBP buffer is from about 1.5% to about 3.5%, preferably from about 2.0% to about 3.0%, more preferred about 2.5%. Typically, the concentration of bovine serum albumin in the SBP buffer is from about 0.5% to about 1.5%, preferably from about 0.75% to about 1.25%, more preferred about 1.0%. Typically, the concentration of phosphate buffer in the SBP buffer is from about 5 mM to about 15 mM, preferably from about 7.5 mM to about 12.5 mM, more preferred about 10 mM. Typically, the pH of the phosphate buffer is from about 7.5 to about 8.5, preferably from about 7.8 to about 8.2, more preferred about 8.0. Typically, in the conditioning step, the solid support is incubated with SBP buffer at room temperature (20-25° C.) for about 15 minutes to about 45 minutes, preferably about 30 minutes.

Subsequent to the conditioning step, the solid support is washed with a buffer referred to herein as “washing buffer.” The washing buffer contains Tris, sodium chloride, magnesium chloride, and a compatible biocide such as Kathon®. Typically, the washing buffer contains from about 25 mM to about 75 mM of Tris, preferably from about 37.5 mM to about 62.5 mM of Tris, more preferred about 50 mM of Tris. Typically, the washing buffer contains from about 100 mM to about 200 mM of sodium chloride, preferably from about 125 mM to about 175 mM of sodium chloride, more preferred about 150 mM of sodium chloride. Typically, the washing buffer contains from about 0.25 mM to about 0.75 mM of magnesium chloride, preferably from about 0.375 mM to about 0.625 mM of magnesium chloride, more preferred about 0.50 mM of magnesium chloride. Typically, the washing buffer contains from about 0.5% to about 1.5% of a compatible biocide such as Kathon®, preferably from about 0.75% to about 1.25% of a compatible biocide such as Kathon®, more preferred about 1.0% of a compatible biocide such as Kathon®. Typically, the pH of the washing buffer is about 7.5 to about 8.5, preferably from about 7.8 to about 8.2, more preferred about 8.0.

Typically, a standard curve is constructed and the concentration of calprotectin is determined by comparison with the standard curve. In one alternative, the standard curve is constructed using a plurality of concentrations of purified calprotectin. In another alternative, the standard curve is constructed using a plurality of concentrations of purified S100A9 such as the recombinant S100A9 described above.

After the samples and standards are added to the solid support, the labelled antibody is added to the solid support; as this is a competitive assay, the labelled antibody and the sample are present simultaneously and are in contact with the solid support to which purified S100A9 polypeptide has previously been bound. After the labelled antibody is added, the solid support, such as the wells of a conventional multiwall ELISA plate, is covered with a suitable covering, such as plastic sheeting or film, tape or a lid and incubated at a suitable temperature, such as room temperature (20-25° C.) for about 10 minutes to about 20 minutes, preferably for about 15 minutes. Typically, the incubation is performed with horizontal shaking at about 500 rpm. Other suitable incubation conditions can be used as is known in the art.

Subsequently, the solid support is washed. Typically, the solid support is washed three times with a washing buffer. If the labelled antibody is labelled with an enzyme that produces a detectable product, a substrate for the enzyme is then added; if the enzyme requires cofactors, such cofactors are also added at that time. Suitable enzymes include, but are not limited to, alkaline phosphatase, horseradish peroxidase, glucose 6-phosphate dehydrogenase, and β-galactosidase. Substrates, as well as any required cofactors for these enzymes are well known in the art.

The reaction of the enzyme with the substrate and any required cofactors is allowed to proceed for a period sufficient to allow the appearance of a sufficient quantity of a detectable product of the enzymatic reaction to detect or determine the quantity of calprotectin in the sample. The appropriate reaction period can be determined by one of ordinary skill in the art, based on factors such as the concentrations of enzyme and substrate, maximum turnover number for the enzyme and the properties of the photometric reader instrument used.

At the end of the reaction period, the enzymatic reaction is stopped, typically by addition of an acid or a solution of sodium hydroxide, depending upon the enzyme used.

Appropriate wavelengths for absorbance determinations for the particular substrates used with combinations of enzymes and substrates are known in the art.

When the assay described above is performed in a conventional ELISA microwell plate, the absorbance can be determined using a conventional microwell ELISA reader. However, absorbance determinations can be performed by other methods known in the art and are not limited to the use of a conventional ELISA microwell reader.

The assay procedure described above is a procedure that employs an enzyme label. However, the assay procedure of the present invention is not limited to an assay employing an enzyme label. For example, a direct label such as a colloidal gold or silver label, stained particles, for instance made of latex or magnetic particles can be employed as described above. In such alternatives, the quantity of the direct label bound to the solid support can be evaluated by instrumentation known in the art. Similarly, if the label is a fluorescent, chemiluminescent, or bioluminescent label, the quantity of the label bound to the solid support can be evaluated by appropriate optical instrumentation for the detection of fluorescence, chemiluminescence, or bioluminescence.

Assays according to the present invention can be used for the detection and quantification of calprotectin in human biological material samples such as a fecal sample, a gastrointestinal tract sample, a blood sample e.g. a serum or plasma sample, a spinal fluid sample, a synovial fluid sample, a saliva sample, a dental crevicular fluid sample, a respiratory or genital tract mucous sample or a urine sample. Samples of animal origin can also be tested if calprotectin or S100A9 in the animal cross-reacts with the human proteins when using the labeled antibody. By experience some antibodies against human calprotectin cross react with calprotectin in primates, dogs and cat. For the purpose of testing for calprotectin or S100A9 in samples from animals, recombinant S100A9 or peptides thereof corresponding to the genes for animal calprotectins can be produced. Antibodies can be raised and labeled as described above for the human protein, and the recombinant proteins or peptides can used for immobilization on solid support and calibrators for competitive immunoassays as described above for the human proteins.

When the sample is a fecal sample or a gastrointestinal tract sample, the sample can be extracted prior to performance of the assay according to the procedure described in U.S. Pat. No. 6,225,072. This extraction procedure comprises: (1) mixing a small amount of sample (preferably 10 to 500 mg and more preferably 20-150 mg, optionally preweighed) with an excess amount of aqueous extraction buffer (preferably in the region of a 50-fold excess (v/v)), comprising at least one dissociating, disaggregating, and/or chelating agent; (2) homogenizing the sample (preferably by vortexing), in a closed tube; (3) separating the solid and liquid material of the dispersion resulting from homogenization of the sample (preferably by centrifugation and additionally or optionally by filtration); and (4) recovering the substantially clear liquid extract resulting from the separation, which contains calprotectin as well as other proteins. A suitable buffer is a citrate buffer with a pH of from about pH 5 to about pH 10. The citrate buffer can be the same citrate buffer described above. In addition to or in stead of citrate, other chelators could be used. The dissociating agent can be an agent such as sodium dodecyl sulfate (SDS) or urea; urea concentrations up to 1 M are particularly suitable. Additionally, the buffer can contain 0.5% to 2% of bovine serum albumin (BSA), optionally in saline. An alternative procedure for preparation of stool extracts comprises a device developed by the Roche Company, Germany. It consists of a 10 ml plastic tube in which is placed a 1 cm wide and 1.5 cm long steel spiral. The latter will contribute to a rapid and efficient disruption of solid particles in the stool sample during vortexing at 1000 rpm for the minimum of three minutes with a suitable extraction buffer. The extract thus prepared can be used without centrifugation.

Generally, the assays according to the present invention, either purified calprotectin or purified S100A9 protein (e.g., recombinant S100A9) can be used as the standard. Often it is preferred to use purified S100A9 protein, particularly recombinant S100A9 protein, as calibrator.

The invented competitive immunoassay has several advantages compared to commonly used sandwich ELISAs on the market
1) They are much quicker, giving results after about 30 minutes allowing quick repeat testing if needed due to technical problems or errors
2) Time delay between addition of sample to the first and last microwell has less effect on the result when compared with sandwich ELISAs
3) They do not carry the risk of a hook affect
4) The assay range is higher so that very few samples with high concentration of calprotectin need to be retested in higher dilution
5) Only one monoclonal antibody selected for proper reactivity against calprotectin in stool extracts
6) Both key reagents, namely the S100A9 protein and the monoclonal, can be provided in large amounts, which will save time and expenses for validation and lot testing. This will result in great economical savings.

In an aspect of the invention a lateral flow test kit is provided. The kit comprises a test line with S100A9 and a control line with labeled antibodies against IgG. In one embodiment the lateral flow test strip is contained in a casing.

Also provided by the present invention is an analyte test element for determining the concentration of calprotectin in a physiological sample is provided. The test element comprising labeled immobilized monoclonal antibodies against S100A9 wherein the calprotectin concentration is determined by the intensity of a signal provided by the label of the labeled immobilized monoclonal antibodies against S100A9 bound to calprotectin from the sample.

The physiological sample used may be a faecal sample, a gastrointestinal tract sample, a blood sample, a serum sample, a plasma sample, or a spinal fluid sample.

The invention is further illustrated by the following non-limiting examples and specific embodiments of the invention.

### Example 1

Microwells of standard 96 well microplate format from Costar, USA, was added 150 μl of a solution of recombinant S100A9 (Delivered from Aldevron Inc., Fargo, N. Dak., USA), 0.8 μg/ml in tris-buffered saline with 1 mM Calcium chloride (TBS-Ca). The wells were covered by plastic tape and left at +5 C for at least 18 hours. Before use, the wells were washed once, 250 μl per well, with TBS-Ca; to each well was then added 250 μl a solution containing 1% BSA, 2.5% sucrose in 10 mM potassium phosphate buffer pH 8 and left at room temperature (22° C.) for 30 minutes. The wells were then washed three times with a buffer containing 50 mM tris, 150 mM NaCl, 0.5 mM magnesium chloride, 1% Kathon and 0.5 ml/l Tween 20, pH 8.0.

In different wells were added 50 μl of qualibrators and samples followed by 50 μl of a HRP-conjugated monoclonal anti-S100A9. The wells were covered by plastic film and incubated with shaking, 500 rpm, for 15 minutes. Subsequently, all wells were washed three times and then added 100 μl HRP TMB substrate. After 20 minutes 100 μl stop solution, 0.2 M sulphuric acid, was added to each well and the colour intensity read at 450 nm in a microwell ELISA reader.

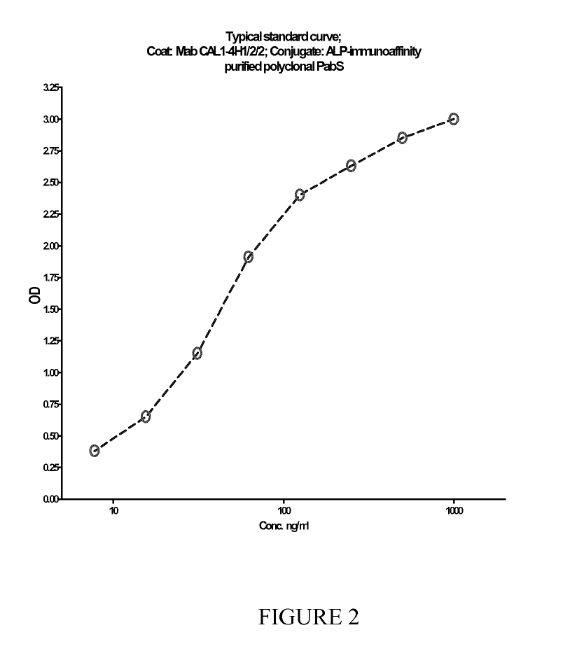

In FIG. 2 is shown the standard curve obtained by this procedure.

### Example 2

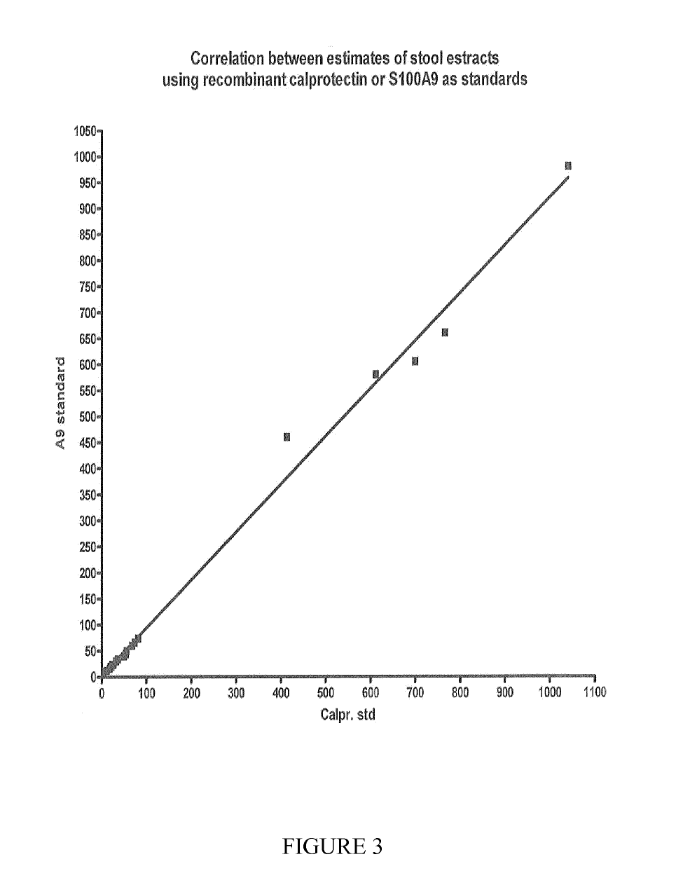

A prerequisite for alternative calprotectin immunoassays is that the results obtained correspond to those from the original ELISA when stool extracts are tested. In FIG. 3 is shown that a satisfactory correlation between the two was found.

### Example 3

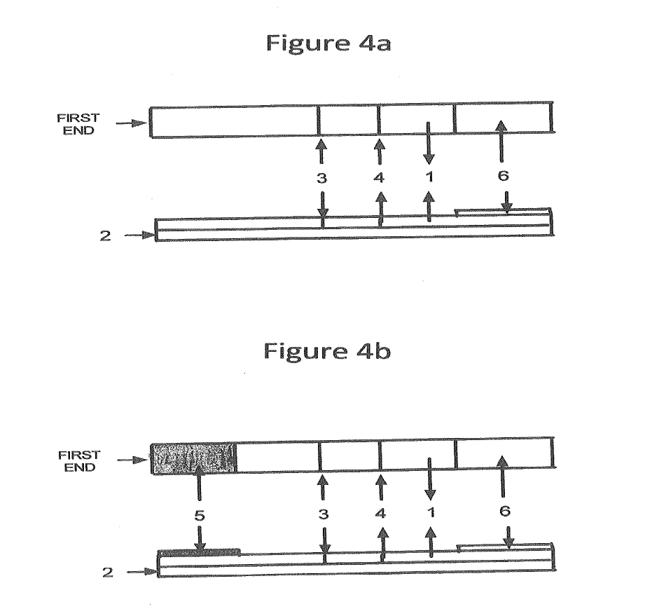

A lateral flow test was set-up as shown in FIGS. 4a and 4b. According to standard methodology well known to people skilled in the art. In brief, it consists of a strip of nitrocellulose, 6 cm long and 0.5 cm wide (marked 1 in the drawing) attached to a rigid, inert plastic membrane (marked 2). Across the strip a solution with 1600 μg/ml S100A9 in PBS was applied as a line in position 3. Similarly, in position 4 a stripe of donkey IgG, 1200 μg/ml in PBS was applied. After incubation at room temperature for one hour, the strip was washed once in PBS and subsequently immersed in PBS with 1% BSA for one hour. Finally the strip was washed three times in PBS and dried. Strips must be kept dry during storage. The test was performed by putting the first end of the strip in vertical position into a microwell containing a mixture of 50 μl sample and 50 μl colloid gold labelled antibodies against S100A9 for the testline and labelled antibodides against donkey IgG for the control line. During a few minutes the sample and antibodies will diffuse upwards into the strip, and the binding of labelled antibodies will bind generate coloured line at position 3 for calprotectin/S100A9 and at position 4 for the control. Optionally, a “sample pad” containing dried labelled antibodies may be attached to the first end of the strip, see FIG. 4b, onto which samples can be applied while keeping the strip horizontally.

The staining of the coloured lines will increase during 30 to 90 minutes, and can be determined by photoelectric scanning and a computer with a suitable program, however, staining intensities may be sufficient for reading after a shorter period, for instance five to 10 minutes.

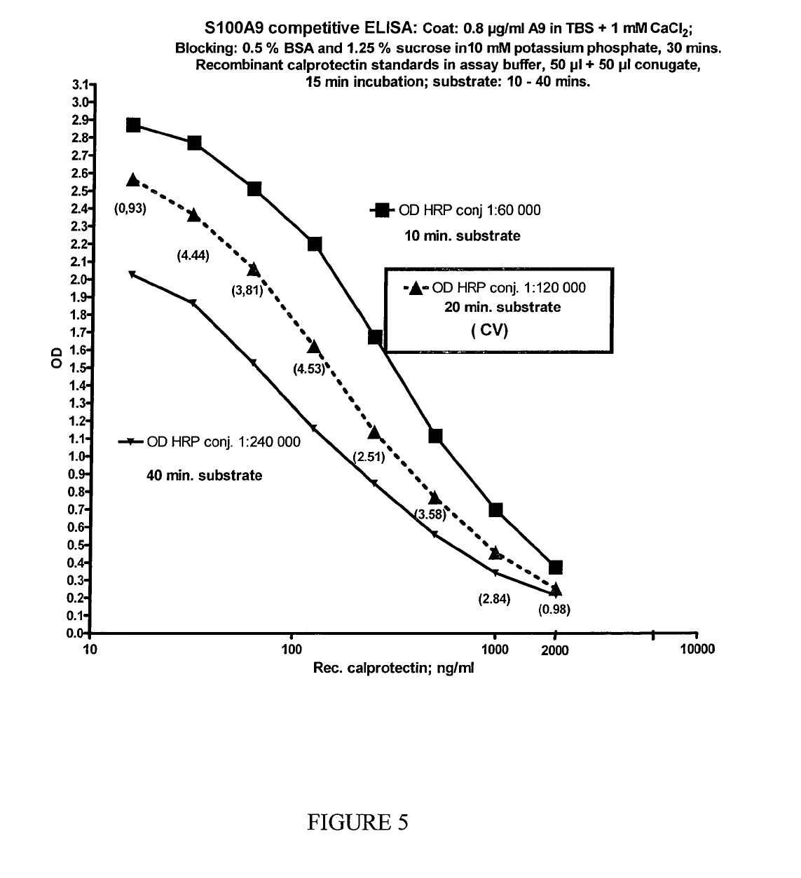

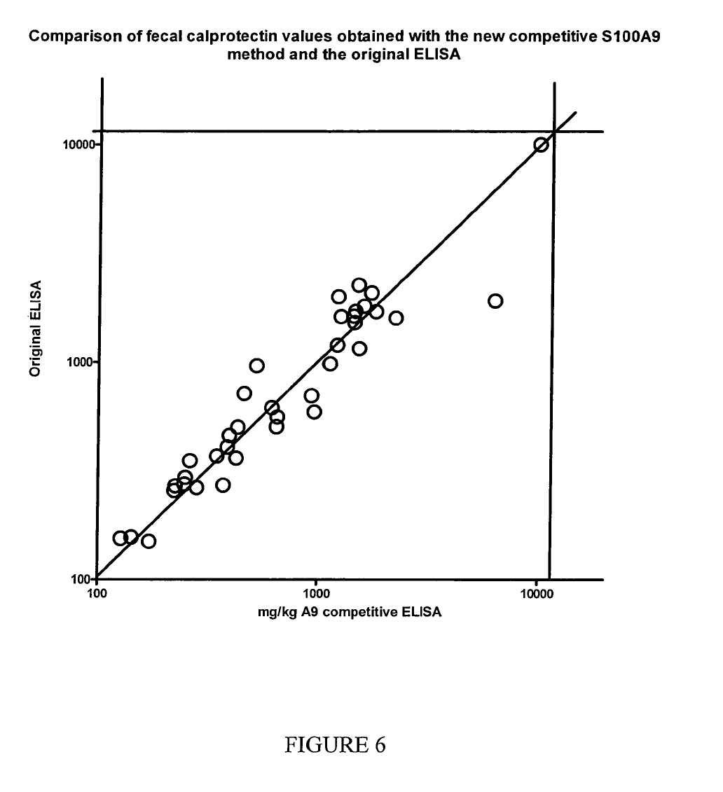

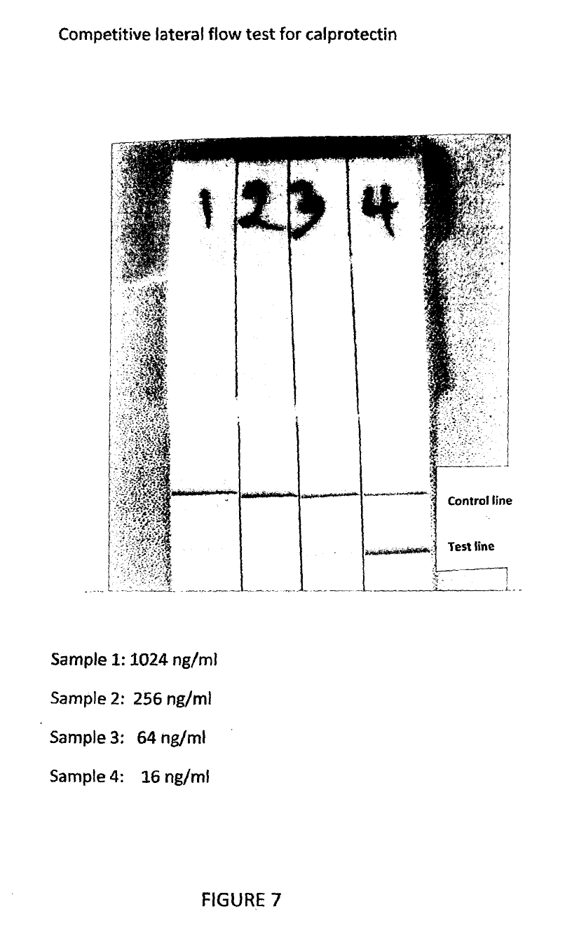

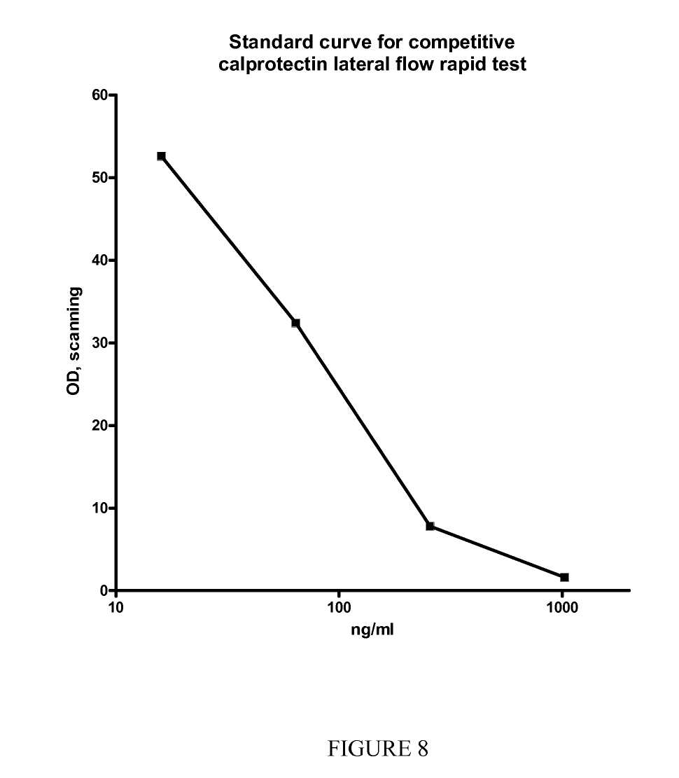

FIG. 5 shows the test and control lines on strips added S100A9 in different amounts together with labelled anti-S100A9 and labelled anti-donkey IgG. The staining intensity of the test line decreases with increasing concentration of S100A9 while the colour of the control line increases with increasing concentration of S100A9.

In other formats of the LFT, the strip can be put in a housing with one or more windows for application of samples and reading of staining intensities of lines. Housing can contain two or more strips for simultaneous testing of several samples or strips intended for simultaneous testing of other proteins, for instance C-reactive protein. Housings can be provided with separate lids or lids hinged to the housing.

1. A method for determining the concentration of calprotectin in a sample comprising the following steps:
i) coating a purified S100A9 or a peptide thereof onto a solid phase,
ii) reacting the S100A9 or peptide coated on the solid phase in step i) with a) a sample containing calprotectin and b) a labelled anti-S 100A9 antibody,
iii) washing of the solid phase after the reaction of any calprotectin in the sample and the labelled anti S100A9 antibody to remove unbound anti-S 100A9 antibody from the solid phase,
iv) determining the quantity of labelled anti-S 100A9 antibody bound to the solid phase to determine the concentration of calprotectin in the sample;
wherein the quantity of S100A9 polypeptide coated onto the solid phase and the quantity of labelled anti-S 100A9 antibody are chosen such that the S100A9 or peptide thereof coated onto the solid phase and any calprotectin in the sample compete for the labelled anti-S100A9 antibody such that the quantity of labelled anti-S100A9 antibody bound to the solid support is related inversely to the concentration of calprotectin in the sample.

2. Methods according to claim 1 wherein the anti-S 100A9 antibody is a monoclonal antibody.

3. Methods according to claim 1 wherein the anti-S 100A9 antibody is a polyclonal antibody.

4. Method according to claim 1 wherein the antibody to S100A9 polypeptide is produced using a recombinant S100A9 polypeptide as antigen.

5. Method according to claim 1 wherein the label of the labelled anti-S 100A9 antibody is an enzyme label.

6. Method according to claim 5 wherein the enzyme of the enzyme-labelled polyclonal antibody is selected from the group consisting of alkaline phosphatase, horseradish peroxidase, glucose 6-phosphate dehydrogenase, and β-galactosidase.

7. Method according to claim 1 wherein calprotectin is coated on the solid phase in step i).

8. Method according to claim 7 wherein the calprotectin is a recombinant calprotectin.

9. Method according to claim 1 wherein free S100A9 is detected.

10. Method according to either of claims 1-5, wherein the sample is a faecal sample, a gastrointestinal tract sample, a blood sample, a serum sample, a plasma sample, or a spinal fluid sample.

11. Method according to either of claims 1-5, wherein the antigen is a mammal S100A9 protein and the labeled antibody is an antibody against a mammal S100A9 protein.

12. Method according to claim 10, wherein the sample to be tested is from a mammal.

13. Lateral flow test kit comprising a test line with S100A9 and a control line with labeled antibodies against IgG.

14. Kit according to claim 13 wherein the lateral flow test strip is contained in a casing.

15. Analyte test element for determining the concentration of calprotectin in a physiological sample comprising labeled immobilized monoclonal antibodies against S100A9 wherein the calprotectin concentration is determined by the intensity of a signal provided by the label of the labeled immobilized monoclonal antibodies against S100A9 bound to calprotectin from the sample.

16. Test element according to claim 15 wherein the physiological sample is a faecal sample, a gastrointestinal tract sample, a blood sample, a serum sample, a plasma sample, or a spinal fluid sample.
